(19) United States
(12) Patent Application Publication
(10) Pub. No.: US 2023/0019922 A1
(43) Pub. Date: Jan. 19, 2023

(54) FRAGMENTATION TIP

(71) Applicant: NIDEK CO., LTD., Gamagori-shi (JP)

(72) Inventors: Naoki OKADA, Gamagori-shi (JP); Takayoshi SHIBATA, Gamagori-shi (JP); Nobuo SUZUKI, Gamagori-shi (JP)

(73) Assignee: NIDEK CO., LTD., Gamagori-shi (JP)

(21) Appl. No.: 17/952,483

(22) Filed: Sep. 26, 2022

Related U.S. Application Data
(63) Continuation of application No. PCT/JP2021/011740, filed on Mar. 22, 2021.

(30) Foreign Application Priority Data
Mar. 24, 2020 (JP) 2020-053423

(51) Int. Cl. A61F 9/007

(52) U.S. Cl. CPC A61F 9/00745; A61B 2217/005

(57) ABSTRACT

A fragmentation tip includes a shaft part and a fragmentation part. The shaft part has a tubular shape having a center axis that matches with a rotational axis of the fragmentation tip. The fragmentation part has a tubular shape and is connected to a distal end portion of the shaft part such that a center axis thereof is inclined to the rotational axis of the fragmentation tip. The fragmentation part and the shaft part form a suction passage therein. The fragmentation part has an annular open end at its distal end. The open end has a linear open end linearly formed when seen from a distal end side in the rotational axis. The linear open end is formed in a portion of the annular open end that is the closest to the rotational axis. The rotational axis passes through a region inside an outer circumference of the annular open end.

### CROSS REFERENCE TO RELATED APPLICATION

This application is a continuation application of International Application No. PCT/JP2021/011740, filed Mar. 22, 2021, which claims priority from Japanese Patent Application No. 2020-053423, filed Mar. 24, 2020. The disclosure of the foregoing applications is hereby incorporated by reference in their entirety.

### BACKGROUND

The present disclosure relates to a fragmentation tip that causes ultrasonic vibration to fragment an eye tissue.

For example, a fragmentation tip has been used to fragment (fracture) and suck (remove) an eye tissue such as an eye lens in which opacity has been caused due to the cataract. The fragmentation tip is configured to fragment an eye tissue using ultrasonic vibration. The eye tissue that has been fragmented and emulsified is sucked through a suction passage within the fragmentation tip.

When an operation is performed using the fragmentation tip, it is preferable that a fragmenting force for fragmenting an eye tissue is larger. For example, Japanese Patent Application Publication No. 2019-084168 discloses a fragmentation tip provided with a shaft part and a fragmentation part. The shaft part is formed in a tubular shape having a rotational axis as a center axis. The fragmentation part is bent relative to the shaft part in a direction of an inclined axis intersecting with the rotational axis and is connected to a distal end of the shaft part. The fragmentation part at the distal end is bent relative to the rotational axis, so that the fragmentation part causes both vibration in a front-rear direction along the rotational axis and torsional vibration around the rotational axis. Thus, the fragmenting force is increased compared to a fragmentation tip (a straight tip) formed linearly toward its distal end.

### SUMMARY

In a configuration in which the fragmentation part at the distal end is bent relative to the rotational axis, as a bending amount of the fragmentation part is larger, a displacement volume of a portion of the fragmentation part distant from the rotational axis is increased, so that the fragmenting force becomes large. However, as the bending amount of the fragmentation tip is larger, the gravity center of the fragmentation part becomes distant from the rotational axis, so that a torsional movement of the whole of the fragmentation tip becomes larger. As a result, bending is caused in an intermediate portion of the fragmentation tip in its axial direction and thus a lateral displacement is caused. The displacement causes negative pressure in an irrigating solution and thus the boiling point of the irrigating solution is decreased. Accordingly, an air bubble (a so-called cavitation) is easily generated. The cavitation generated in the intermediation portion of the fragmentation part is easy to damage an eye tissue (for example, the cornea or the iris) into which the fragmentation tip is inserted. When the bending amount of the fragmentation part is decreased, the generation of the cavitation in the intermediate portion is suppressed, however the fragmenting force is decreased. Thus, the conventional fragmentation tip is difficult to increase the fragmenting force at the distal end while suppressing the generation of the cavitation in the intermediate portion in the axial direction.

Embodiments of the broad principles derived herein provide a fragmentation tip (phacoemulsification tip, or phaco tip) that is capable of suppressing generation of a cavitation in an intermediate portion in an axial direction and is capable of increasing a fragmenting force at a distal end against an eye tissue.

Embodiments provide a fragmentation tip that is configured to cause ultrasonic vibration so as to fragment an eye tissue. The fragmentation tip includes: a shaft part formed in a tubular shape, the shaft part having a center axis that matches with a rotational axis of the fragmentation tip, and a fragmentation part formed in a tubular shape and connected to a distal end portion of the shaft part in a state in which a center axis of the fragmentation part is inclined to the rotational axis. The fragmentation part and the shaft part form a suction passage therein. The fragmentation part has an annular open end at its distal end. The annular open end has a linear open end linearly formed when seen from a distal end side in the rotational axis, the linear open end being formed in a portion of the annular open end that is the closest to the rotational axis. The rotational axis passes through a region inside an outer circumference of the annular open end when seen from the distal end side in the rotational axis.

According to the fragmentation tip of the present disclosure, the generation of the cavitation in the intermediate portion in the axial direction is suppressed and the fragmenting force at the distal end against an eye tissue is increased.

### BRIEF DESCRIPTION OF DRAWINGS

FIG. 1 is a right side view (a partial sectional view) of a US handpiece 2 to which a fragmentation tip 1 is mounted.

FIG. 2 is a perspective view of a fragmentation tip 1A of a first embodiment seen from a right upper side thereof.

FIG. 3 is a right side view of a portion near a distal end of the fragmentation tip 1A of the first embodiment.

FIG. 4 is a perspective view of the portion near the distal end of the fragmentation tip 1A of the first embodiment seen from the right upper side thereof.

FIG. 5 is a front view of the portion near the distal end of the fragmentation tip 1A of the first embodiment seen from a distal end side in a rotational axis R.

FIG. 6 is a front view of a portion near a distal end of a fragmentation tip 1B of a second embodiment seen from a distal end side in a rotational axis R.

FIG. 7 is a front view of a portion near a distal end of a fragmentation tip 1C of a third embodiment seen from a distal end side in a rotational axis R.

FIG. 8 is a front view of a portion near a distal end of a fragmentation tip 1D of a fourth embodiment seen from a distal end side in a rotational axis R.

FIG. 9 is a perspective view illustrating a displacement volume of a conventional fragmentation tip 100 as a comparative example.

FIG. 10 is a perspective view illustrating a displacement volume of the fragmentation tip 1A of the first embodiment.

FIG. 11 is a perspective view illustrating a displacement volume of the fragmentation tip 1B of the second embodiment.

FIG. 12 is a perspective view illustrating a displacement volume of the fragmentation tip 1C of the third embodiment.

### DETAILED DESCRIPTION

A fragmentation tip exemplarily described in the present disclosure includes a shaft part and a fragmentation part. The shaft part is formed in a tubular shape. The shaft part has a center axis that matches with a rotational axis of a whole of the fragmentation tip. The fragmentation part is formed in a tubular shape. The fragmentation part is connected to a distal end portion of the shaft part in a state in which a center axis of the fragmentation part is inclined to the rotational axis of the whole of the fragmentation tip. The fragmentation part and the shaft part form a suction passage therein. The fragmentation part has an annular open end at its distal end. The annular open end has a linear open end (straight open end) linearly formed when seen from a distal end side in the rotational axis. The linear open end is formed in a portion of the annular open end that is the closest to the rotational axis (the portion of the annular open end may include the rotational axis). The rotational axis passes through a region inside an outer circumference of the annular open end when seen from the distal end side in the rotational axis.

A displacement volume of the linear open end disposed in the annular open end located at the distal end of the fragmentation part when the linear open end rotates around the rotational axis is larger than a displacement volume of an open end formed in an arc shape curved to protrude in a direction getting far away from the center axis of the fragmentation part. Thus, the ultrasonic vibration on the fragmentation part having the linear open end causes large fragmenting force to fragment an eye tissue. Further, the linear open end is disposed in a portion of the open end that is the closest to the rotational axis. Thus, the energy of rotation of the linear open end around the rotational axis hardly causes the torsion of the whole fragmentation tip which leads to bending, so that the cavitation is hardly generated in an intermediate portion in the axial direction. Further, the rotational axis passes through the region inside the outer circumference of the annular open end when seen from the distal end side. Thus, a bending amount of the fragmentation part relative to the shaft part is small, compared to a configuration in which the rotational axis passes outside the outer circumference of the open end. Accordingly, the fragmentation tip exemplarily described in the present disclosure can realize that the generation of the cavitation in the intermediate portion in the axial direction is suppressed and the fragmenting force against an eye tissue is increased.

The length of the linear open end in its longitudinal direction may be equal to or longer than one-half of the inner diameter of the shaft part when the open end of the fragmentation part is seen from the distal end side in the rotational axis. In this case, the displacement volume when the linear open end rotates around the rotational axis is increased, compared to a configuration in which the length of the linear open end is shorter than one-half of the inner diameter of the shaft part. Further, as described above, even in a case in which the length of the linear open end is set to be longer, the linear open end is located near the rotational axis, so that the rotation of the linear open end hardly causes the cavitation. Consequently, an eye tissue is appropriately fragmented.

The length of the linear open end in the longitudinal direction is preferably equal to or more than two-third of the inner diameter of the shaft part, more preferably more than the inner diameter of the shaft part.

The linear open end may be located on the rotational axis of the fragmentation tip. In this case, compared to a configuration in which the linear open end is offset from the rotational axis, a possibility can be further reduced that the energy of the rotation of the linear open end causes the cavitation in the intermediate portion in the axial direction. Consequently, an eye tissue is further appropriately fragmented.

However, even in a configuration in which the linear open end is offset from the rotational axis, in a case in which the linear open end is close to the rotational axis, the generation of the cavitation in the intermediate portion in the axial direction is suppressed and the fragmenting force against an eye tissue is increased.

The center axis of the fragmentation part inclined to the rotational axis may orthogonally intersect with a longitudinal direction of the linear open end when seen from the distal end side in the rotational axis. In this case, the annular open end is easily set in a symmetrical shape relative to a plane passing both of the rotational axis and the center axis of the fragmentation part. Thus, the fragmented eye tissue is easily and smoothly sucked through the open end. Here, the configuration of “the center axis orthogonally intersects with the longitudinal direction of the linear open end” is not limited to a configuration in which the center axis “strictly” orthogonal to the longitudinal direction of the linear open. That is, the configuration of “the center axis orthogonally intersects with the longitudinal direction of the linear open end” also includes a configuration in which the center axis of the fragmentation part substantially orthogonally intersects with the linear open end. Also in such a case, the fragmented eye tissue is easily and smoothly sucked through the open end.

The annular open end located at the distal end of the fragmentation part may be formed in a polygonal shape having a plurality of linear sides including the linear open end when seen from the distal end side in the rotational axis. In this case, the open end has a linear portion other than the linear open end that is close to the rotational axis. The displacement volume of the linear portion in the open end is larger than the displacement volume of a portion curved to protrude in a direction getting far away from the rotational axis. Thus, the fragmenting force against an eye tissue is further increased.

A portion other than the linear open end in the annular open end located at the distal end of the fragmentation part may be curved in an arc shape to protrude in a direction getting far away from the rotational axis when seen from the distal end side in the rotational axis. In this case, when the fragmentation part rotates around the rotational axis, a resistance of fluid in an eye is hardly applied to the portion curved in an arc shape. Thus, the energy of rotation of the curved portion that is distant from the rotational axis hardly causes the torsion of the whole fragmentation tip, so that the cavitation is further hardly caused in the intermediation portion in the axial direction.

An opening area of the annular open end located at the distal end of the fragmentation part may be larger than an opening area of the shaft part when seen from the distal end side in the rotational axis. In this case, an eye tissue (for example, a nucleus of an eye lens) is easily retained by the open end of the fragmentation part having a large opening area. Consequently, the retained eye tissue is appropriately and easily fragmented and sucked.

US Handpiece

Typical embodiments of the present disclosure are now described with reference to the drawings. First, a US handpiece 2 to which a fragmentation tip 1 (1A, 1B, 1C, or 1D) of the present embodiment is mounted is described. The US handpiece 2 is configured to cause ultrasonic vibration on the fragmentation tip 1 mounted to a distal end of the US handpiece 2 so as to fragment (fracture) and emulsify an eye tissue (a nucleus of an eye lens of a subject eye in which opacity has been caused due to the cataract) and suck and remove the fragmented eye tissue.

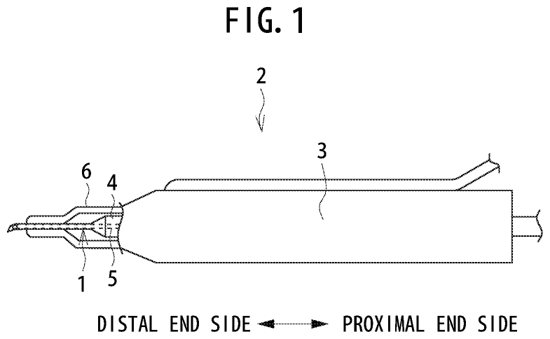

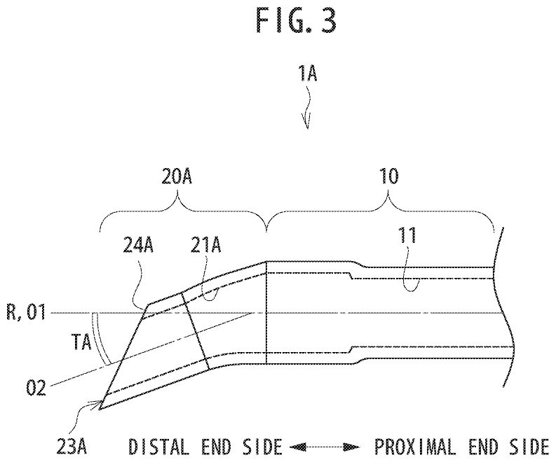

As shown in FIG. 1, the US handpiece 2 includes a handpiece body 3 and a sleeve 6. The handpiece body 3 includes a horn 4 and a suction passage 5. The fragmentation tip 1 is detachably mounted to a distal end of the horn 4. The horn 4 amplifies ultrasonic vibration caused by a transducer (not shown) and transmits the amplified ultrasonic vibration to the fragmentation tip 1 mounted to the distal end thereof. The suction passage 5 extends rearward from the distal end of the horn 4 through an inside of the horn 4. When the fragmentation tip 1 is mounted to the distal end of the horn 4, the suction passage 5 of the handpiece body 3 is communicated with a suction passage 11 (see FIG. 3) of the fragmentation tip 1. An eye tissue fragmented by the fragmentation tip 1 and an irrigating solution (for example, normal saline solution or physiological saline solution) supplied into the eye are sucked from the inside of the eye to the proximal end of the fragmentation tip 1 through the suction passage 5, by a suction force generated by a suction device (not shown). The sleeve 6 is formed in a tubular shape (for example, a hollow cylindrical shape) and is detachably mounted to a distal end portion of the handpiece body 3. The sleeve 6 is formed of a flexible material such as silicone resin. The sleeve 6 covers the proximal end side of the fragmentation tip 1 in a state in which the distal end of the fragmentation tip 1 is exposed.

Fragmentation Tip

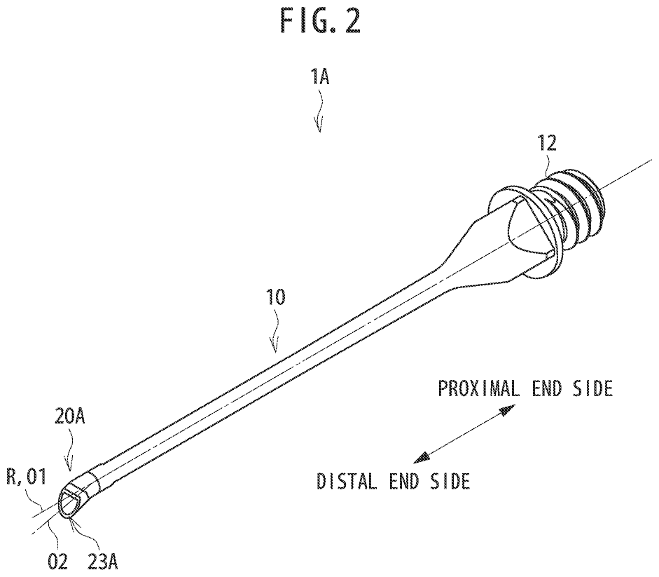

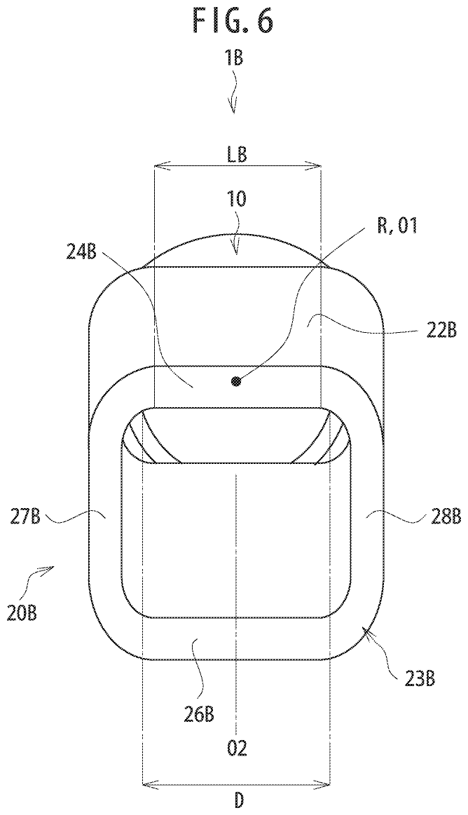

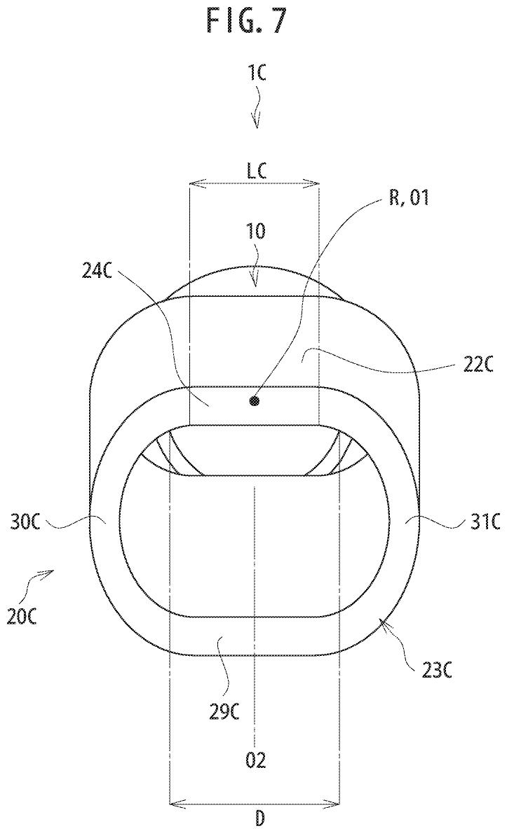

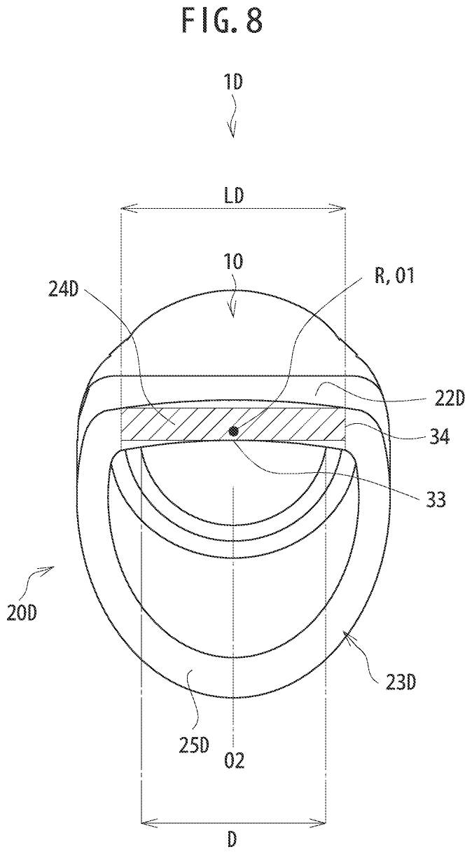

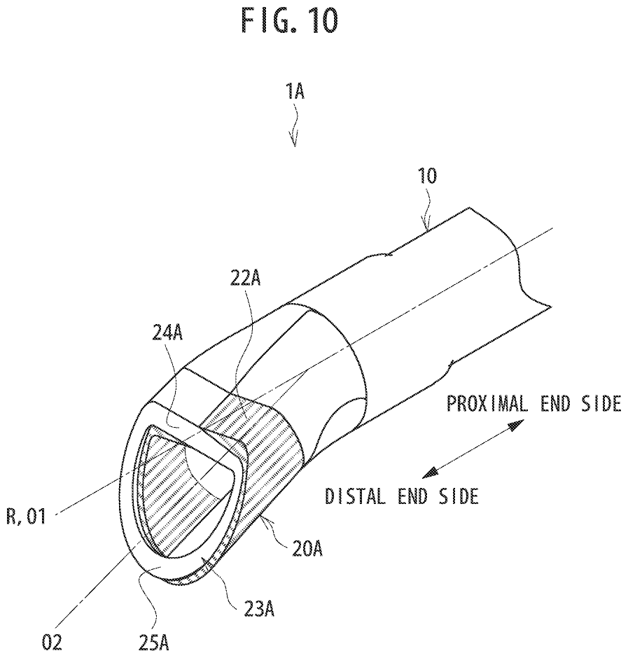

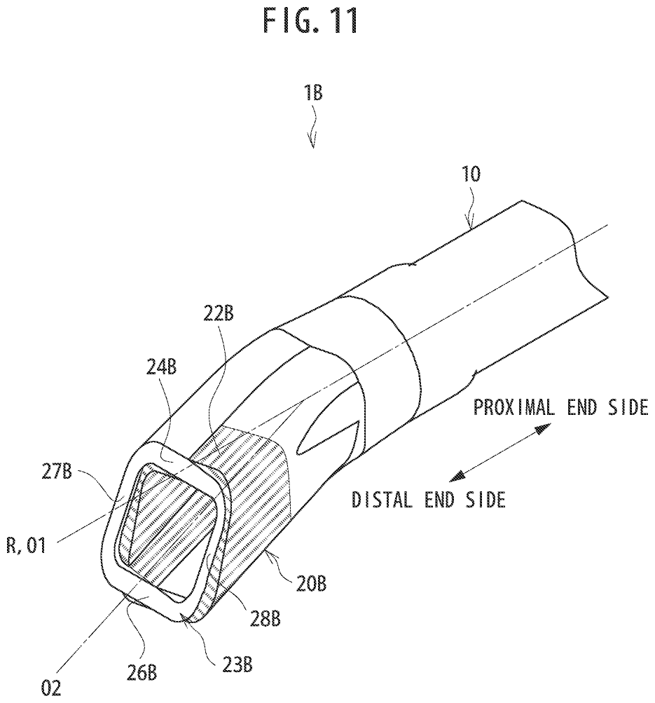

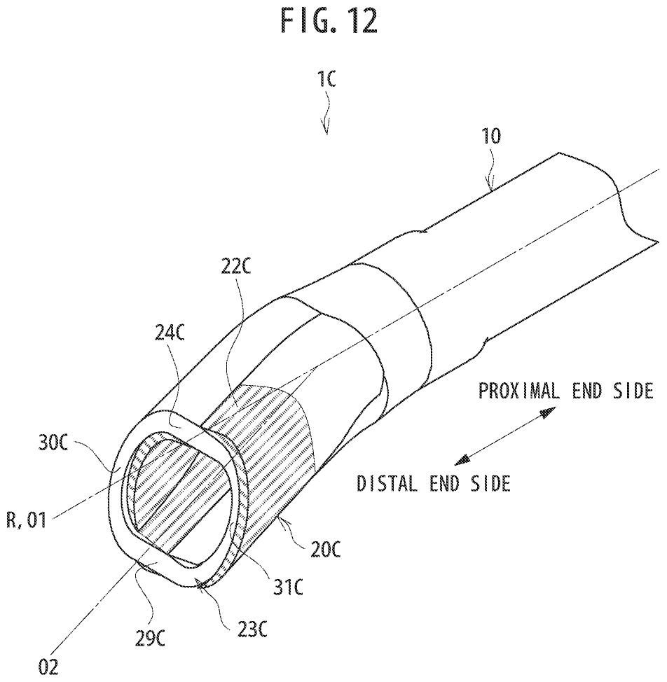

The fragmentation tips 1 (1A, 1B, C and 1D) are now described. In the following description, the fragmentation tip 1A (see FIG. 2 to FIG. 5, and FIG. 10), the fragmentation tip 1B (see FIG. 6 and FIG. 11), the fragmentation tip 1C (see FIG. 7 and FIG. 12), and the fragmentation tip 1D (see FIG. 8) are exemplarily described in the first to fourth embodiments, respectively.

In the four fragmentation tips 1A, 1B, 1C and 1D exemplarily described in the present embodiments, configurations of fragmentation parts 20A, 20B, 20C and 20D are different from each other, however a configuration of a shaft part 10 and a configuration of a center axis O2 of each of the fragmentation parts 20A, 20B, 20C and 20D inclined to a rotational axis R are common in all embodiments. Thus, a configuration that is common in the four fragmentation tips 1 (1A, 1B, 1C and 1D) is firstly described using the fragmentation tip 1A of the first embodiment. The fragmentation tip 1 is formed of a material having an appropriate rigidity and heat resistance (for example, titanium alloy).

As shown in FIG. 2 to FIG. 5, the fragmentation tip 1 includes the shaft part 10 and the fragmentation part 20. The shaft part 10 is formed in a tubular shape (typically, a hollow cylindrical shape). Thus, the shaft part 10 is an elongate hollow tube extending linearly. A center axis O1 of the tubular shaft part 10 matches with the rotational axis R of the fragmentation tip 1. When the ultrasonic vibration is caused on the fragmentation tip 1, the shaft part 10 reciprocatingly rotates within a specified angular range around the rotational axis R. A sectional shape orthogonal to the center axis O1 of the shaft part 10 of the present embodiment is in symmetry relative to the center axis O1. Thus, even when the torsional vibration is caused on the fragmentation tip 1, water pressure of the irrigating solution is hardly applied to the shaft part 10.

As shown in FIG. 3, a suction passage 11 is formed in the tubular shaft part 10. The suction passage 11 allows an eye tissue fragmented by the fragmentation part 20A and an irrigating solution supplied into an eye to pass therethrough. As shown in FIG. 2, a mount part 12 configured to detachably mount the fragmentation tip 1 to the distal end of a horn 4 (see FIG. 1) of the US handpiece 2 is formed in a proximal portion of the shaft part 10. The suction passage 11 of the shaft part 10 allows an end portion of the shaft part 10 at a distal end side and the mount part 12 of the shaft part 10 at a proximal end side to communicate with each other.

As shown in FIG. 2 to FIG. 5, the fragmentation part 20A is formed in a tubular shape. As shown in FIG. 2, the fragmentation part 20A is connected to the distal end portion of the shaft part 10 in a state in which the center axis O2 of the fragmentation part 20A is inclined to the rotational axis R of the fragmentation tip 1 (which matches with the center axis O1 of the shaft part 10) by an angle TA. The fragmentation part 20A of the present embodiment is inclined such that the center axis O2 is getting far in a downward direction in FIG. 2 from the rotational axis R, toward the distal end of the fragmentation part 20A. A suction passage 21A is formed in the fragmentation part 20A. The suction passage 21A allows a fragmented eye tissue and the irrigating solution to pass therethrough. The suction passage 21A of the fragmentation part 20A is communicated with the suction passage 11 of the shaft part 10. Thus, the fragmentation part 20A and the shaft part 10 form a suction passage therein.

Since the fragmentation part 20A is bent relative to the rotational axis R, when the fragmentation tip 1 reciprocatingly rotates around the rotational axis R, the fragmentation part 20A reciprocatingly rotates (torsionally vibrates) within a specified angular range. An angle of the torsional vibration of the fragmentation part 20A is larger than an angle of the reciprocating rotation of the shaft part 10. As a result, a fragmenting force against an eye tissue is increased, compared to a fragmentation tip formed linearly toward its distal end.

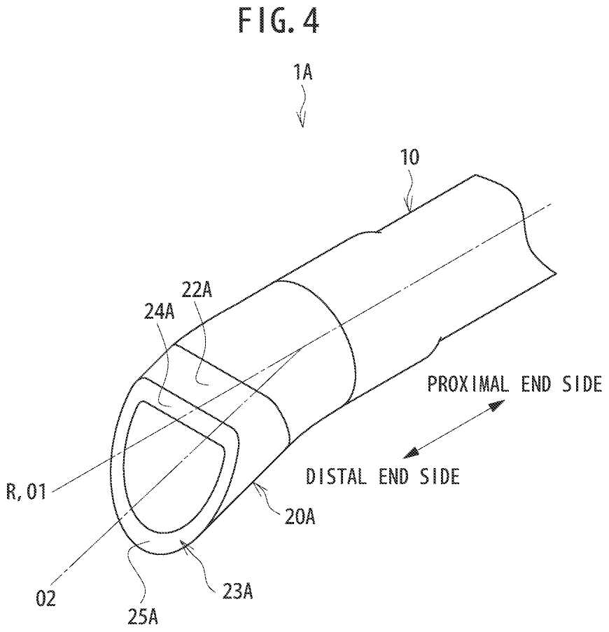

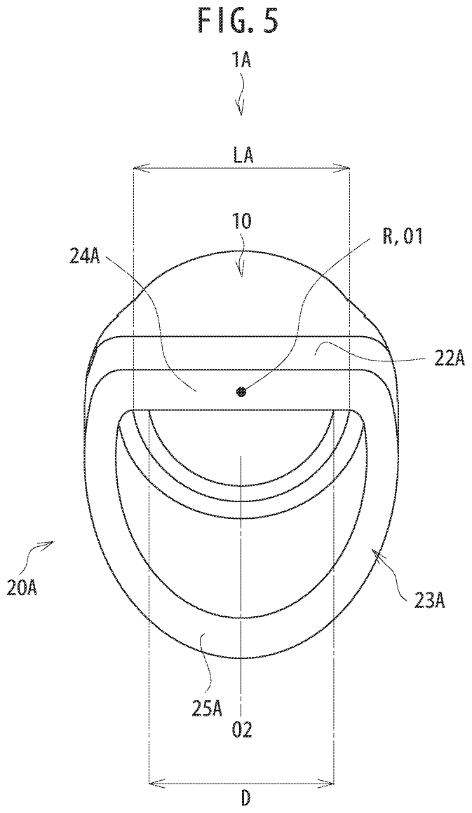

As shown in FIG. 4 and FIG. 5, an annular open end 23A is located on the distal end of the fragmentation part 20A. The open end 23A gets into contact with an eye tissue while torsionally vibrating, so that the open end 23A serves as a fragmenting part that fragments the eye tissue.

As shown in FIG. 3 and FIG. 4, since the center axis O2 of the fragmentation part 20A is inclined to the rotational axis R, the annular open end 23A has a portion that is close to the rotational axis R and a portion that is distant from the rotational axis R. An end portion that is the most distant from the rotational axis R (a lower end portion in FIG. 3 and FIG. 4) in the open end 23A of the fragmentation part 20A protrudes from an end portion that is the closest to the rotational axis R (an upper end portion in FIG. 3 and FIG. 4), toward the distal end side in a direction along the rotational axis R. Thus, when the open end 23A is seen in a direction orthogonal to both of the rotational axis R and the center axis O2 of the fragmentation part 20A (namely, seen from a front surface side of the paper of FIG. 3), an angle of the end portion that is the most distance from the rotational axis R (namely, an angle of the distal end portion) is a sharp angle. The energy of the torsional vibration is large in the sharp-angle portion that is the most distant from the rotational axis R. Thus, compared to a configuration in which an angle of an end portion that is the most distant from the rotational axis R is an obtuse angle (90 degrees or more), the fragmenting force against an eye tissue caused by the fragmentation part 20A is further increased.

The above-described configuration of the fragmentation tip 1 exemplarily described in the first embodiment is common to the second to fourth embodiments. Accordingly, in the second to fourth embodiments described below, the detailed description of the shaft part 10 or the like is omitted.

### First Embodiment

The detailed configuration of the fragmentation part 20A of the fragmentation tip 1A of the first embodiment is now described. As shown in FIG. 4 and FIG. 5, a linear open end 24A (straight open end) that is linearly formed when seen from the distal end side in the rotational axis R (namely, seen from a front surface side of the paper of FIG. 5) is disposed at least in a portion that is the closest to the rotational axis R of the fragmentation tip 1A, in the annular open end 23A located at the distal end of the fragmentation part 20A. A displacement volume of a portion near the linear open end 24A when rotating around the rotational axis R is larger than that of a portion near the open end formed in an arc shape curved to protrude in a direction getting far away from the center axis O2 of the fragmentation part 20A, which will be described in detail later. Thus, when the fragmentation part 20A having the linear open end 24A ultrasonic-vibrates, an eye tissue is fragmented by a large fragmenting force. Further, the linear open end 24A is disposed in the portion of the open end 23A that is the closest to the rotational axis R. Thus, the energy of rotation of the linear open end 24A around the rotational axis R hardly causes the torsion in the whole fragmentation tip 1A, so that the cavitation is hardly generated in the shaft part 10 that is an intermediate portion in the axial direction.

As shown in FIG. 4 and FIG. 5, an outer peripheral surface of the fragmentation part 20A, extending rearward from the linear open end 24A is formed as a flat plane 22A extending in parallel to the center axis O2 from the linear open end 24A. The flat plane 22A is inclined to the rotational axis R not to be in parallel to the rotational axis R. As a result, the linear open end 24A is disposed near the rotational axis R, so that the generation of the cavitation in the intermediate portion in the axial direction is suppressed. Further, the displacement volume of the fragmentation part 20A is also increased by disposing the flat plane 22A, which will be described in detail later.

As shown in FIG. 5, when the open end 23A is seen from the distal end side in the rotational axis R, the length LA of the linear open end 24A in a longitudinal direction (a left-right direction in FIG. 5) is equal to or longer than one-half of the inner diameter D of the shaft part 10. Thus, the displacement volume when the linear open end 24A rotates around the rotational axis R is increased, compared to a configuration in which the length of the linear open end 24A is shorter than one-half of the inner diameter D of the shaft part 10. Also in a case in which the length of the linear open end 24A is set to be longer, the linear open end 24A is located near the rotational axis R. Accordingly, the rotation of the linear open end 24A hardly causes the cavitation in the intermediate portion in the axial direction.

In the fragmentation tip 1A of the first embodiment, when the open end 23A is seen from the distal end side in the rotational axis R, the length LA of the linear open end 24A in the longitudinal direction is equal to or longer than two-third of the inner diameter D of the shaft part 10, more specifically longer than the inner diameter D of the shaft part 10. Thus, the displacement volume of a portion near the linear open end 24A is further increased.

As shown in FIG. 5, when the open end 23A is seen from the distal end side in the rotational axis R, the rotational axis R passes through a region inside an outer circumference of the annular open end 23A disposed at the distal end of the fragmentation part 20A. Thus, compared to a configuration in which the rotational axis R passes outside the open end 23A, a bending amount of the fragmentation part 20A relative to the shaft part 10 (namely, at least one of: the inclined angle TA of the fragmentation part 20A relative to the shaft part 10; and the length of the fragmentation part 20A) becomes small. Thus, an eye tissue is fragmented by the fragmentation part 20A having the linear open end 24A using a large fragmenting force while suppressing the generation of the cavitation in the intermediate portion in the axial direction due to the large bending amount of the fragmentation part 20A.

Specifically, in this embodiment, as shown in FIG. 5, the linear open end 24A which is a part of the annular open end 23A is located on the rotational axis R. Thus, compared to a configuration in which the linear open end 24A is offset from the rotational axis R, a possibility can be reduced that the energy of the rotation of the linear open end 24A causes the cavitation in the intermediate portion in the axial direction.

As shown in FIG. 5, when the open end 23A is seen from the distal end side in the rotational axis R, the center axis O2 of the fragmentation part 20A inclined to the rotational axis R orthogonally intersects with the longitudinal direction of the linear open end 24A (the left-right direction in FIG. 5). In this case, the whole of the annular open end 23A is easily set in a symmetrical shape relative to a plane passing both of the rotational axis R and the center axis O2 of the fragmentation part 20A (namely, a symmetrical shape in the left-right direction in FIG. 5). Thus, the fragmented eye tissue is easily and smoothly sucked through the open end 23A.

As shown in FIG. 5, when the open end 23A is seen from the distal end side in the rotational axis R, a portion of the annular open end 23A other than the linear open end 24A is formed as a curved open end 25A having an arc shape curved to protrude in a direction getting far away from the rotational axis R. When the fragmentation part 20A rotates around the rotational axis R, a resistance of fluid (in this embodiment, an irrigating solution) in an eye is hardly applied to the curved open end 25A. Thus, the energy of rotation of the curved open end 25A that is distant from the rotational axis R hardly causes torsion of the whole of the fragmentation tip 1A, so that the cavitation is further hardly caused in the intermediation portion in the axial direction.

As shown in FIG. 5, when the open end 23A is seen from the distal end side in the rotational axis R, an opening area of the annular open end 23A is larger than an opening area of the shaft part 10. Thus, an eye tissue is easily retained by the open end 23A having a large opening area. Consequently, the retained eye tissue is appropriately and easily fragmented and sucked.

As shown in FIG. 3 and FIG. 4, the linear open end 24A in the open end 23A of the fragmentation part 20A is located at the proximal end side relative to the distal end (the left end portion in FIG. 3, or the left-lower end portion in FIG. 4) of the open end 23A. More specifically, the linear open end 24A is disposed at the most proximal side in the annular open end 23A. In this case, the angle of the end portion in the open end 23A that is the most distant from the rotational axis R (namely, the potion at the most distal end side) is a sharp angle. In the sharp-angle portion that is distant from the rotational axis R, the energy of the torsional vibration is large. Thus, the fragmenting force caused by the fragmentation part 20A against an eye tissue is further increased. A configuration in each of the following second to fourth embodiments is similar to the above-described configuration in which the linear open end 24A is located at the proximal end side relative to the distal end of the open end 23A. Accordingly, in the following embodiments, the description relating to the positional relation between the distal end of the open end 23A and the linear open end 24A is omitted.

The second to fourth embodiments are now described. The configuration and the effect similar to those in the first embodiment are briefly described, or the description thereof is omitted.

### Second Embodiment

A configuration of a fragmentation part 20B of the fragmentation tip 1B of the second embodiment is described with reference to FIG. 6. As shown in FIG. 6, a linear open end 24B that is linearly formed when seen from the distal end side in the rotational axis R of the fragmentation tip 1B (namely, seen from a front surface side of the paper of FIG. 6) is disposed at least in a portion that is the closest to the rotational axis R, in an annular open end 23B located at the distal end of the fragmentation part 20B. Thus, a fragmenting force against an eye tissue can be enhanced while suppressing the generation of the cavitation in the intermediate portion in the axial direction.

An outer peripheral surface of the fragmentation part 20B, extending rearward from the linear open end 24B is formed as a flat plane 22B extending in parallel to the center axis O2 from the linear open end 24B. The flat plane 22B is inclined to the rotational axis R not to be in parallel to the rotational axis R. As a result, the linear open end 24B is disposed near the rotational axis R.

When the open end 23B is seen from the distal end side in the rotational axis R, the length LB of the linear open end 24B in the longitudinal direction (a left-right direction in FIG. 6) is equal to or longer than one-half (specifically, two-third) of the inner diameter D of the shaft part 10. Thus, the displacement volume of a portion near the linear open end 24B is increased.

When the open end 23B is seen from the distal end side in the rotational axis R, the rotational axis R passes through a region inside an outer circumference of the annular open end 23B disposed at the distal end of the fragmentation part 20B. Specifically, the linear open end 24B is located on the rotational axis R. Accordingly, the generation of the cavitation in the intermediate portion in the axial direction is further suppressed.

When the open end 23B is seen from the distal end side in the rotational axis R, the center axis O2 of the fragmentation part 20B inclined to the rotational axis R orthogonally intersects with the longitudinal direction of the linear open end 24B (the left-right direction in FIG. 6). Thus, the whole of the annular open end 23B is easily set in a symmetrical shape relative to a plane passing both of the rotational axis R and the center axis O2 of the fragmentation part 20B. Accordingly, the fragmented eye tissue is easily and smoothly sucked through the open end 23B.

When the open end 23B is seen from the distal end side in the rotational axis R, the annular open end 23B is formed in a polygonal shape with a plurality of linear sides (straight sides) including the linear open end 24B. Specifically, the open end 23B in the second embodiment includes the linear open end 24B, a linear facing open end 26B disposed to face the linear open end 24B to extend in parallel to the linear open end 24B, and linear connecting open ends 27B, 28B each connecting an end of the linear open end 24B and an end of the facing open end 26B. Thus, the shape of the open end 23B in the second embodiment seen from the distal end side in the rotation axis R is a generally rectangular shape. In a case in which the open end 23B is a polygonal shape, the open end 23B has a linear portion other than the linear open end 24B that is closer to the rotational axis R. The displacement volume of the linear portion in the open end 23B is larger than the displacement volume of a portion curved to protrude in a direction getting far away from the rotational axis R. Thus, the fragmenting force against an eye tissue can be further enhanced.

The open end seen from the distal end side in the rotation axis R may be formed in a polygonal shape other than a rectangular shape (for example, a triangular shape or a pentagonal shape).

### Third Embodiment

A configuration of a fragmentation part 20C of the fragmentation tip 1C of the third embodiment is described with reference to FIG. 7. As shown in FIG. 7, a linear open end 24C that is linearly formed when seen from the distal end side in the rotational axis R of the fragmentation tip 1C (namely, seen from a front surface side of the paper of FIG. 7) is disposed at least in a portion that is the closest to the rotational axis R in an annular open end 23C located at the distal end of the fragmentation part 20C. Thus, a fragmenting force against an eye tissue can be enhanced while suppressing the generation of the cavitation in the intermediate portion in the axial direction.

An outer peripheral surface of the fragmentation part 20C, extending rearward from the linear open end 24C is formed as a flat plane 22C extending in parallel to the center axis O2 from the linear open end 24C. The flat plane 22C is inclined to the rotational axis R not to be in parallel to the rotational axis R. As a result, the linear open end 24C is disposed near the rotational axis R.

When the open end 23C is seen from the distal end side in the rotational axis R, the length LC of the linear open end 24C in the longitudinal direction (a left-right direction in FIG. 7) is equal to or longer than one-half (specifically, two-third) of the inner diameter D of the shaft part 10. Thus, the displacement volume of a portion near the linear open end 24C is increased.

When the open end 23C is seen from the distal end side in the rotational axis R, the rotational axis R passes through a region inside an outer circumference of the annular open end 23C disposed at the distal end of the fragmentation part 20C. Specifically, the linear open end 24C is located on the rotational axis R. Accordingly, the generation of the cavitation in the intermediate portion in the axial direction is further suppressed.

When the open end 23C is seen from the distal end side in the rotational axis R, the center axis O2 of the fragmentation part 20C inclined to the rotational axis R orthogonally intersects with the longitudinal direction of the linear open end 24C (the left-right direction in FIG. 7). Thus, the whole of the annular open end 23C is easily set in a symmetrical shape relative to a plane passing both of the rotational axis R and the center axis O2 of the fragmentation part 20C. Accordingly, the fragmented eye tissue is easily and smoothly sucked through the open end 23C.

When the open end 23C is seen from the distal end side in the rotational axis R, the annular open end 23C includes a linear side other than the linear open end 24C and a curved side formed in an arc shape curved to protrude in a direction getting far away from the center axis O2. Specifically, the open end 23C in the third embodiment includes the linear open end 24C, a linear facing open end 29C disposed to face the linear open end 24C to extend in parallel to the linear open end 24C, and arc-shaped connecting open ends 30C, 31C each connecting an end of the linear open end 24C and an end of the facing open end 29C. In the third embodiment, the linear side and the curved side are disposed in a portion in the open end 23C other than the linear open end 24C, so that the fragmenting force is increased by the linear side and the generation of the cavitation is suppressed by the curved side.

### Fourth Embodiment

A configuration of a fragmentation part 20D of the fragmentation tip 1D of the fourth embodiment is described with reference to FIG. 8. As shown in FIG. 8, a linear open end 24D that is disposed in a portion that is the closest to the rotational axis R of the fragmentation tip 1D, in an annular open end 23D located at the distal end of the fragmentation part 20C is formed in not a completely linear shape but a slightly curved shape. In this manner, also in a case in which the linear open end 24D is slightly curved, a fragmenting force against an eye tissue can be enhanced while suppressing the generation of the cavitation in the intermediate portion in the axial direction.

As one example, relating to the portion of the open end 23D that is the closest to the rotation axis R, a side curved to protrude (an upper side in FIG. 8) is defined as a convex side, while a side opposite to the convex side (a lower side in FIG. 8) is defined as a concave side. A portion in an edge part of the concave side that is the closest to the convex side is defined as a reference portion 33. When the portion of the open end 23D that is the closest to the rotational axis R is seen from the distal end side in the rotational axis R, a maximum rectangular 34 is set to pass the reference portion 33 and have a longitudinal direction orthogonal to the center axis O2. In a case in which an area of the maximum rectangular 34 (the area hatched by oblique lines in FIG. 8) is 75% or more of an area of a portion in the open end 23D that is the closest to the rotational axis R and overlaps with the rectangular 34 in the longitudinal direction, the generation of the cavitation is suppressed and the fragmenting force is enhanced. In such a case, the linear open end 34D is deemed to be formed in a portion that is the closest to the rotational axis R.

Similarly, in a case in which an outer peripheral surface of the fragmentation part 20D, extending rearward from the linear open end 24D is formed as a flat plane 22D, the flat plane 22D need not be necessarily formed in a complete flat plane, and thus the outer peripheral surface may be curved as needed so as not to deteriorate the above-described effect.

When the open end 23D is seen from the distal end side in the rotational axis R, the length LD of the linear open end 24D in the longitudinal direction is equal to or longer than one-half of the inner diameter D of the shaft part 10, more specifically longer than the inner diameter D of the shaft part 10. Further, when the open end 23D is seen from the distal end side in the rotational axis R, the rotational axis R passes through a region inside an outer circumference of the annular open end 23D. Specifically, the linear open end 24D is located on the rotational axis R. When the open end 23D is seen from the distal end side in the rotational axis R, the center axis O2 of the fragmentation part 20D inclined to the rotational axis R orthogonally intersects with the longitudinal direction of the linear open end 24D. The above-described configuration is similar to that in each of the first to third embodiments. The shape of the portion other than the linear open end 24D in the open end 23D in the fourth embodiment is similar to that in the first embodiment. However, the shape of the portion other than the linear open end 24D in the open end 23D in the fourth embodiment may be set to be similar to that in the second or third embodiment.

Comparing of Displacement Volumes

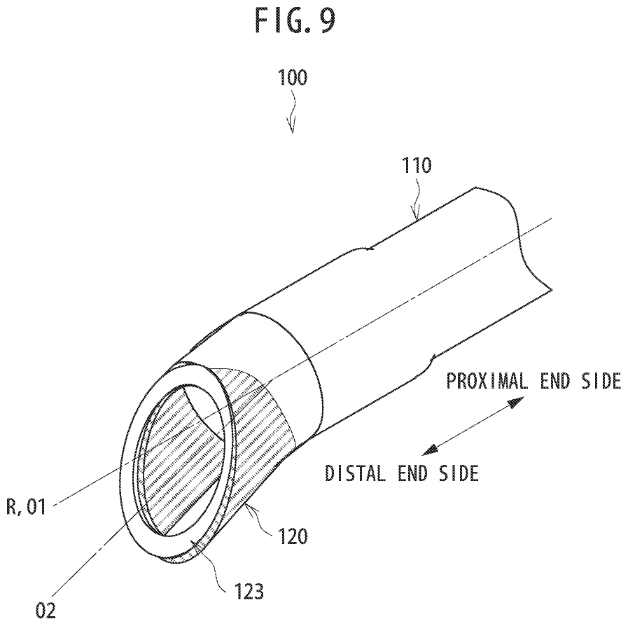

Comparing results between a displacement volume of a conventional fragmentation tip 100 (see FIG. 9) that is a comparative example, and a displacement volume of each of the fragmentation tips 1A, 1B and 1C of the first to third embodiments are described with reference to FIG. 9 to FIG. 12. In the fragmentation tip 100 of the comparative example shown in FIG. 9, a linear open end is not formed in an open end 123 at the distal end of a fragmentation part 120, which is different from the fragmentation tips 1 in the above-described first to fourth embodiments. However, a configuration other than the linear open end in the fragmentation tip 100 of the comparative example (for example, a configuration of the shaft part 10, and a configuration of the center axis O2 of the fragmentation part 120 inclined to the rotational axis R) is similar to those of the fragmentation tips 1 in the above-described first to fourth embodiments.

In the simulation described below, the displacement volume of each of the fragmentation tip 100 of the comparative example (see FIG. 9), the fragmentation tip 1A of the first embodiment (see FIG. 10), the fragmentation tip 1B of the second embodiment (see FIG. 11) and the fragmentation tip 1C of the third embodiment (see FIG. 12) when the ultrasonic vibration with amplitude of 170 μm is caused thereon, was measured based on a three-dimension model, and then the displacement volume is illustrated by hatching in each figure. Further, simulation of the displacement volume of each fragmentation tip was executed using a three-dimension CAD software.

It can be obviously recognized by comparing the figures that the displacement volume (the volume of the hatched portion) of each of the fragmentation tips 1A, 1B and 1C shown in FIG. 10 to FIG. 12 is larger than the displacement volume of the fragmentation tip 100 of the comparative example shown in FIG. 9. Actually, the displacement volume of the fragmentation tip 100 of the comparative example shown in FIG. 9 is 0.052 mm³, while the displacement volume of the fragmentation tip 1A of the first embodiment shown in FIG. 10 is 0.098 mm³, the displacement volume of the fragmentation tip 1B of the second embodiment shown in FIG. 11 is 0.0194 mm³, and the displacement volume of the fragmentation tip 1C of the third embodiment shown in FIG. 12 is 0.0194 mm³. It is found that, by disposing each of the linear open end 24A, 24B and 24C in the portion that is the closest to the rotation axis R in the open end of the fragmentation part as described above, the displacement volume is increased and the fragmenting force against an eye tissue is enhanced, compared to the fragmentation tip 100 of the comparative example.

In each of the second and third embodiments in which the linear side is disposed in the open end of the fragmentation part in addition to the linear open end, the displacement volume is increased compared to that in the first embodiment. Accordingly, it is found that, by disposing the linear side other than the linear open end in the open end of the fragmentation part, the fragmenting force against an eye tissue is further enhanced.

The apparatus and methods described above with reference to the various embodiments are merely examples. It goes without saying that they are not confined to the depicted embodiments. While various features have been described in conjunction with the examples outlined above, various alternatives, modifications, variations, and/or improvements of those features and/or examples may be possible. Accordingly, the examples, as set forth above, are intended to be illustrative. Various changes may be made without departing from the broad spirit and scope of the underlying principles. Further, only a part of the techniques in the above-described embodiments may be employed.

What is claimed is:

1. A fragmentation tip configured to cause ultrasonic vibration to fragment an eye tissue, the fragmentation tip comprising:
a shaft part formed in a tubular shape, the shaft part having a center axis that matches with a rotational axis of the fragmentation tip; and
a fragmentation part formed in a tubular shape and connected to a distal end portion of the shaft part in a state in which a center axis of the fragmentation part is inclined to the rotational axis, the fragmentation part and the shaft part forming a suction passage therein,
wherein:
the fragmentation part has an annular open end at its distal end,
the annular open end has a linear open end linearly formed when seen from a distal end side in the rotational axis, the linear open end being formed in a portion of the annular open end that is the closest to the rotational axis, and
the rotational axis passes through a region inside an outer circumference of the annular open end when seen from the distal end side in the rotational axis.

2. The fragmentation tip as defined in claim 1, wherein the linear open end is located on the rotational axis.

3. The fragmentation tip as defined in claim 1, wherein the center axis of the fragmentation part inclined to the rotational axis orthogonally intersects with a longitudinal direction of the linear open end when seen from the distal end side in the rotational axis.

4. The fragmentation tip as defined in claim 1, wherein the annular open end located at the distal end of the fragmentation part is formed in a polygonal shape having a plurality of linear sides including the linear open end when seen from the distal end side in the rotational axis.

5. The fragmentation tip as defined in claim 1, wherein a portion other than the linear open end in the annular open end located at the distal end of the fragmentation part is curved in an arc shape when seen from the distal end side in the rotational axis.
